(19) United States
(12) Patent Application Publication
(10) Pub. No.: US 2016/0195512 A1
(43) Pub. Date: Jul. 7, 2016

(54) METHOD FOR DETERMINING THE CLEANING PERFORMANCE OF FORMULATIONS

(71) Applicant: L'AIR LIQUIDE, SOCIETE ANONYME POUR L'ETUDE ET L'EXPLOITATION DES PROCEDES GEORGES CLAUDE, Paris (FR)

(72) Inventors: Judith HEIDEL, Pinneberg (DE); Katrin STEINHAUER, HAMBURG (DE); Heike JORDAN, Henstedt-Ulzburg (DE)

(21) Appl. No.: 14/911,294

(22) PCT Filed: Sep. 4, 2014
(86) PCT No.: PCT/EP2014/068810

§ 371 Date: Feb. 10, 2016

(30) Foreign Application Priority Data
Sep. 13, 2013 (DE) 10 2013 218 448.4

(51) Int. Cl. G01N 33/483

(52) U.S. Cl. CPC G01N 33/4833

(57) ABSTRACT

A method for determining the cleaning performance of a formulation, wherein
a) the formulation is provided,
b) a test body is brought into contact with the formulation, the test body being contaminated with a protein-containing test contaminant,
c) the contaminated test body is left in contact with the formulation in order to clean the contaminated test body,
d) the cleaned test body is rinsed,
e) if necessary the rinsed test body is dried,
f) if necessary the test contaminant remaining on the test body is evaluated in terms of quality, and
g) the test contaminant remaining on the test body is analyzed in terms of quantity, the quantitative analysis of the remaining test contaminant including the removal of the remaining test contaminant from the test body.

The present invention relates to a method for determining the cleaning performance of formulations with regard to protein-containing contaminants.

According to the prior art methods are known for evaluating the performance of formulations during the removal of protein-containing contaminants (cf. including DE 10 2006 006 765 A1). Furthermore, WO 97/27482 A1 discloses a synthetic test contaminant for testing the effectiveness of machine cleaning methods.

The test contaminant of WO 97/27482 A1 contains fibrin and/or a fibrin precursor. The test contaminant coagulates and can be used for testing the effectiveness of cleaning methods with regard to blood impurities. Once the test body has been subjected to the cleaning methods to be tested, according to the teaching of WO 97/27482 A1 residues of the test contaminant are detected on the test body, for example by means of an optical evaluation. The optical evaluation typically involves staining protein residues remaining on the test body by means of a detection reaction. Alternatively, WO 97/27482 A1 proposes hydrolysing proteins adhering to the test body and analysing the resulting amino acids as products of the hydrolysis. For the quantitative detection it proposes including radioactive tracers in the proteins of the test contaminant, such as for example ^{99m}Tc, and then measuring the gamma radiation coming from the test body.

The methods according to WO 97/27482 are expensive and potentially associated with health risks (radioactivity) and are also not sufficiently reliable.

Consequently, the objective was to find a method by means of which, in a simple manner and without any potential risk to health, the cleaning performance of different formulations could be evaluated, both in terms of quantity and quality, and also in such a reliable manner that it is also possible to distinguish between very effective cleaning formulations in a reproducible manner.

It has proved to be surprising that this problem can be addressed by a method, in which

a) the formulation is provided,
b) a test body is brought into contact with the formulation, the test body being contaminated with a protein-containing test contaminant,
c) the contaminated test body is left in contact with the formulation in order to clean the contaminated test body,
d) the cleaned test body is rinsed,
e) if necessary the rinsed test body is dried,
f) if necessary the test contaminant remaining on the test body is evaluated in terms of quality and
g) the test contaminant remaining on the test body is analysed in terms of quantity, the quantitative analysis of the remaining test contamination including the removal of the remaining test contaminant from the test body.

The method according to the invention addresses the aforementioned problem and is characterised in particular in that it is possible to differentiate in a reproducible manner between very effective cleaning formulations, the performance of which differs in detail however in the removal of protein.

In the method according to the invention in step a) the formulation is provided, typically as an aqueous solution of a concentrate.

Then in step b) a test body is brought into contact with the formulation, the test body being contaminated with a protein-containing test contaminant. Such test bodies contaminated with protein-containing contaminants are known. Preferably, the test contaminant is a synthetic test contaminant, preferably a test contaminant which comprises one or more components selected from a group including fibrin, a fibrin precursor, haemoglobin and albumin.

Preferably, the protein-containing test contaminant is a synthetic test contaminant, preferably a synthetic test contaminant which comprises at least two different protein components, preferably at least three different protein components.

It is preferable that the protein components of the synthetic test contaminant, as already mentioned, are selected from a group comprising fibrin, a fibrin precursor, haemoglobin and albumin.

The term “synthetic” means that the test contaminant does not consist of a single whole blood.

It is thus possible in one embodiment to mix the whole blood of at least two different mammals, in order to obtain a synthetic test contaminant. In a further alternative a synthetic test contaminant is used which is obtained by mixing at least two different (i.e. previously isolated) blood components; said blood need not then necessarily originate from different mammals.

Typically, the test bodies are made of metal, i.e. not glass, which enables particularly good reproducible results for the quantification g).

The contaminated test body is left in contact with the formulation in step c) in order to clean the contaminated test body. The contact c) can be for example a static or a dynamic contact between the formulation and the contaminated test body, i.e. the formulation is either left in or moved into contact with the contaminated test body.

Once the contaminated test body has been left in contact with the formulation and is thus at least partly cleaned, the cleaned test body is rinsed in step d), the rinsing preferably including rinsing with water and in particular the rinsing is preferably with water.

After the rinsing d) the rinsed test body is dried if necessary (and preferably), the drying e) preferably being performed at a temperature of in the range of 10 to 40° C., preferably in a range of from 15 to 30° C., in particular about 25° C. Particularly preferably, the drying is performed in air at ambient temperature.

After the optional drying e) the test contaminant remaining on the test body is evaluated qualitatively if necessary. This evaluation is preferably an optical evaluation on a scale with at least 10 levels, preferably at least 12 levels, in particular at least 16 levels.

Steps e) and f) are optional respectively.

In a particularly preferred embodiment, which is also explained in more detail in connection with method I in the examples, a cleaning standard series is established and then the optical evaluation is performed in that a comparison is made directly with this cleaning standard series and/or with photos of this standard series.

In particular, this optical evaluation of the test contaminant remaining on the test body is an evaluation in four rough levels, wherein the four rough levels each comprise four finer levels, and wherein the uncleaned test body with the protein-containing test contaminant represents an additional level and the test body without a protein-containing test contaminant likewise represents an additional level (i.e. such a standard series 18 comprises a total of 18 levels).

Preferably, during the qualitative evaluation f) the test contaminant remaining on the test body is evaluated optically and photographed.

It should also be noted that the optional qualitative evaluation f) in all embodiments of the method according to the invention preferably does not include a chemical detection reaction, i.e. the protein-containing test contaminant is evaluated directly and also photographed in the state in which it was cleaned off by contact with the formulation and remained on the test body after rinsing and drying.

After the optional qualitative evaluation f) in the method according to the invention a quantitative analysis of the remaining test contaminant g) is performed, wherein the quantitative analysis of the remaining test contaminant includes the removal of the remaining test contaminant from the test body.

Preferably, the remaining test contaminant is quantified photometrically during the quantitative analysis g) after the removal of the test contaminant. It is particularly preferable, in the method according to the invention to quantify the protein contained in the test contaminant and removed therefrom as a part of the test contaminant photometrically.

Alternatively, the quantitative analysis g) includes gel electrophoresis, such as SDS-Page.

In all of the embodiments of the invention it is preferable if the said photometric determination of protein contained in the test contaminant and removed therefrom is performed in a buffered solution with a pH of 5 to 9, in particular with a pH of 6 to 8.

During the described removal of the test contaminant remaining on the test body, preferably said removal includes rinsing the remaining test contaminant from the test body by means of one or more aqueous solutions.

Preferably, the aqueous solutions used for rinsing include both an alkaline and an acidic aqueous solution. It is preferred in this case that the test contaminant remaining on the test body in step g) is firstly rinsed by an alkaline aqueous solution and is then rinsed by an acidic aqueous solution, in order in the end to obtain an essentially pH neutral solution of the test contaminant remaining on the test body.

In all of the embodiments of the invention it is preferred that the removal of the test contaminant remaining on the test body in step g) includes mechanical cleaning. For example, it is possible that the test body with the test contaminant remaining thereon is brought into contact with glass beads (and preferably shaken), in particular whilst the test body with the remaining test contaminant is being brought into contact with one or more aqueous solutions during the removal thereof.

It is also preferable if the photometric quantification of the protein contained in the remaining test contaminant and removed therefrom is performed by a triphenylmethane dye.

Coomassie Brilliant Blue G-250 is the preferred triphenylmethane dye. The photometric quantification is most preferably a Bradford quantification. Tests for such a quantification are commercially available (e.g. the Roti-Nanoquant test of the company Carl Roth GmbH+ Co. KG, Karlsruhe, Germany).

The invention also relates to a kit for performing the method according to the invention, which comprises
(i) a set of instructions with a description of the method,
(ii) one or more solutions for removing the remaining contaminant,
(iii) one or more mechanical means for removing the remaining contaminant, the preferred means being glass beads, and
(iv) if necessary a standard series of cleaned test bodies for the qualitative evaluation f), or an image of the standard series.

The advantages of the invention, in particular the improved reproducible differentiation between different cleaning formulations, are described in particular in the following examples. The quantities relate to weight, unless otherwise indicated.

### EXAMPLES

### Method I

### Determining the Cleaning Performance by Means of TOSI Test Bodies and Quantitative Bradford Protein Determination

The method is used for determining the cleaning performance of cleaning solutions for preparing medical instruments (IDM=instrument disinfectants). TOSI (Test Object Surgical Instruments) are used as the test bodies, the test contaminant of which correlates to human blood.

The test can be performed as a static test in order to simulate the behaviour of the manual preparation of instruments, or as a dynamic test in order to represent the cleaning capability in machine preparation.

In this method the visual evaluation after the cleaning attempt follows the quantitative determination of the protein film remaining on the test body with the reagent Roti-Nanoquant. Based on the Bradford protein assay [Bradford, M., (1976) Anal. Biochem. 72:248-254. Niess, U., (2004) J Bacteriol. 186:3640-3648] in this case the proteins are identified by the dye Coomassie Brilliant Blue G 250.

The concentration of the cleaning solution, the quality of the water used (demineralised, softened, mains water etc.), the duration of the cleaning test and the temperature of the test are selected respectively according to the use of the product in practice.

Required materials, chemicals and implements
Magnetic stirrer, possibly with connected water bath
Thermostat
Beakers, tall, 250 ml and 100 ml
Magnetic stirring rod
Weight rings
Umbilical cord clamp
Device for hanging up the umbilical cord clamp
Eppendorf Pipette P5000 and P1000 with corresponding pipette tips
pH meter
Small tube 15 ml with lid
Shaker
Tweezers
400 ml beaker with demineralised water
Digital camera
TOSI test body (order no. 8302, BAG Health Care, Lich, Germany)
Timer
Glass beads
Disposable cuvettes
Cuvette paddles (for mixing)
Disposable pipettes
NaOH solution, 0.5 mol/l
HCl solution, 0.5 mol/l
Buffer pH 7.00 (Merck)
Albumin Serum Fraction V (Serva)
Roti-Nanoquant (Roth)
Photometer (590 nm and 450 nm)

From the Roti-Nanoquant solution a 20% solution is produced in demineralised water. Said dilution can be kept refrigerated for a week.

Performing the Cleaning Tests

a) Static Cleaning Test

The beakers (100 ml, tall) are filled foam-free with about 100 ml of the test solution to be tested. The TOSI test bodies are placed into the solution with tweezers with the test contaminant layer at the top. At the end of the test period the TOSI test bodies are removed from the solution with the tweezers and rinsed by immersing and swirling in VE-water. The TOSI-test bodies are then dried upright in air.

Afterwards an optical evaluation is performed of the TOSI test bodies into groups and possibly subgroups by comparison with the previously determined comparison TOSI test bodies (standard). For documentation the TOSI-Test bodies are photographed with a digital camera. The images are copied later into the evaluation sheets. Each TOSI test body can now be evaluated analytically by the Bradford method.

b) Dynamic Cleaning Test

The beakers (250 ml, tall) are filled with 200 ml of the cleaning solution to be tested, provided with a magnetic stirring rod. When using a water bath the beakers are made heavier with a lead ring. Afterwards they are put on the stirrer (usually step 3) at room temperature or put on the stirrer in the water bad at test temperature.

At the beginning of the test the TOSI test bodies are removed from the packaging and from the plastic mounts, placed into a suitable holder (e.g. umbilical cord clamp) and suspended centrally in the beaker with the cleaning solution. At the end of the test period the TOSI test bodies are removed from the solution with the tweezers and rinsed by immersing and swirling in VE-water. The TOSI test bodies then dry upright in the air.

Afterwards an optical evaluation of the TOSI test bodies is performed into groups and/or subgroups in comparison with the relevant standard TOSI test bodies defined before the start of the test. For documentation the TOSI test bodies are photographed with a digital camera. The images are later copied into the evaluation sheets. Each TOSI test body can then be evaluated analytically by the Bradford method.

Definition of the Cleaning Standard Series for the Qualitative Evaluation

For the reproducible optical evaluation of the TOSI test bodies a cleaning standard series is produced. For this cleaned test bodies have been allocated into groups and subgroups.

With a 0.5% solution of a commercially available alkaline enzymatic cleaning agent a cleaning series was carried out with different removal times of the TOSI test bodies: the removal times were after 10 s, 20 s, 30 s, 40 s, 50 s, 60 s, 70 s, 80 s, 90 s, 100 s, 110 s, 120 s, 240 s, 270 s, 330 s, 360 s and 600 s.

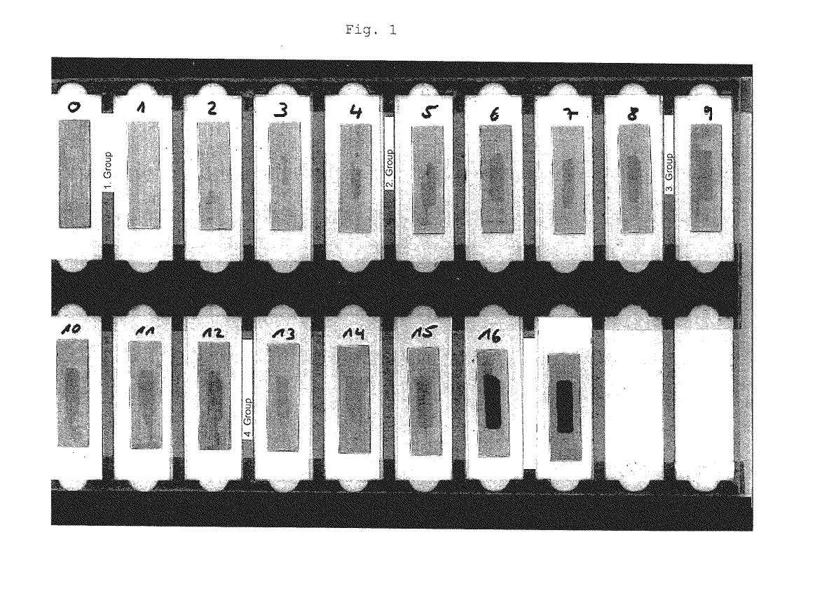

For the clear traceability of the appearance several subgroups were formed (see Table 1 and FIG. 1).

TABLE 1

| Group | Subgroup |
|---|---|
| A (no residue) | 0 |
| B (little residue) | 1-4 |
| C (almost complete area with residue) | 5-8 |
| D (complete area with residue, slightly yellowish) | 9-12 |
| E (almost complete residue, velvety coating) | 13-16 |
| F (uncleaned test body with test contaminant) | 17 |

From the subjective assessment a very good qualitative evaluation is achieved by means of the cleaning standard series, which thus, independently of the assessing person, is always the same and thus easily comparable.

Bradford quantitative protein assay with Roti-Nanoquant 5 ml 0.5 M NaOH solution with about 10-15 glass beads are put into a 15 ml tube, the sealed tubes are kept at about 55° C. in a water bath, a TOSI test body is put into a tube and shaken forcefully with the shaker until all of the residue is in solution.

5 ml 0.5 M HCl solution is added to the respective tube with the 0.5 M NaOH-solution, the TOSI test body and the glass beads, and the TOSI test body is rinsed with the 5 ml 0.5 M HCl solution; the test body is then taken out of the tube and disposed of.

The solution from the tube is adjusted by the addition of 5 ml buffer solution pH 7.0 to pH 7.0±0.1. For the blank value 5 ml 0.5 M NaOH solution, 5 ml 0.5 M HCl solution and 5 ml buffer solution pH 7.0 are mixed in a 30 ml glass and adjusted to a pH value of 7.0±0.1.

Then 400 μl of the adjusted solution (or the blank value) and 1600 μl of the 20% Roti-Nanoquant solution are placed into a cuvette and mixed. After 5 min reaction time the samples are measured photometrically. For this firstly a zero adjustment is carried out with water at 590 nm, then the blank value and sample are also measured at 590 nm. Afterwards, the zero adjustment and measurements are carried out at 450 nm.

Evaluation:

Protein μg/ml=(Eₛₐₘₚₗₑ₅₉₀ₙₘ/Eₛₐₘₚₗₑ₄₅₀ₙₘ−E_{blindvalue590nm}/E_{blindvalue450 nm})/increase in levels

Calibration of the Quantification of Protein

To produce a calibration level different BSA concentrations are used (BSA: Bovine Serum Albumin). For this a stock solution is used with a concentration of 400 μg/ml BSA in VE-water. From this solutions are produced with a concentration of 10 μg/ml and 100 μg/ml. From these two solutions the dilution series is produced (see Table 2).

TABLE 2

| BSA | | μl fully |
|---|---|---|
| [μg/ml] | μl from BSA dilution | desalinated water |
| 0 | — | 400 |
| 1 | 40 μl from 10 μg/ml | 360 |
| 2.5 | 100 μl from 10 μg/ml | 300 |
| 5 | 200 μl from 10 μg/ml | 200 |
| 10 | 40 μl from 100 μg/ml | 360 |
| 25 | 100 μl from 100 μg/ml | 300 |
| 50 | 200 μl from 100 μg/ml | 200 |
| 75 | 300 μl from 100 μg/ml | 100 |
| 100 | 200 μl from 400 μg/ml | 600 |

The calibrating solutions are produced in a cuvette. For this 400 μl of the corresponding BSA concentration solution (see Table 2) is displaced by 1600 μl of the 20% Roti-Nanoquant solution and mixed with a cuvette paddle.

After 5 min. reaction time in the cuvette at the photometer firstly a zero adjustment with water is performed at 590 nm and afterwards the calibrating solutions are measured. The calibrating solutions and also the zero adjustment with water are also measured at the wavelength 450 nm.

The quotient of the two extinctions (590 nm/450 nm) is formed and with the quotient the calibration line is produced.

### Method II

### Cleaning Test with Coagulated Sheep's Blood

Target and Application Area

This comparison method is used for determining the cleaning performance of cleaning and disinfectant solutions with coagulated sheep's blood as the test contaminant.

Supply and Production of Test Contaminant

Sheep's blood in Na-Heparin, uninhibited
Sheep's blood in Na-Heparin 10 IU/ml, Fiebig Nährstofftechnik (Idstein, Germany)
Protamine sulfate ME, 1000 IU/ml, MEDA, 5 ml ampoules
Production

9 ml sheep's blood is stirred with 1 ml VE-water and 0.15 ml protamine sulphate in a small beaker with a magnetic stirring rod. The application time is about 7 minutes, then the blood coagulates.

Required Materials

General:
Slide made of glass with matt edge
Measuring cylinder, 100 ml, filled with acetone
Measuring cylinder, 100 ml, filled with petrol ether, two
Tweezers
Analysis scales with a precision of at least ±0.1 mg
Desiccator with silica gel
Stopwatch
Beakers, tall, 100 ml

Method

Preparation of the Slide

The slides are numbered and cleaned. Then weighing out is performed on the analysis scales.

Application of the Blood Contaminant

The cleaned slides are laid out next to one another. About 55-60 μl blood is pipetted on and spread with an impregnated brush wiped off on the edge of the glass.

After a drying time of about 1 hour at RT the slides are stored overnight in the desiccator for the complete drying of the test bodies by means of silica gels.

Quantity of Blood Contaminant

After drying overnight only those slides are used for the remaining test which have an even film of blood contaminant. The slides are weighed. The weight difference from the empty slides indicates the quantity of the test contaminants. The following quantities of blood contaminant per slide are aimed for:

| | Blood | |
|---|---|---|
| Quantity Ø | 10.0 | mg |
| Quantity min. (−20%) | 8.0 | mg |
| Quantity max. (+20%) | 12.0 | mg |

Test Conditions

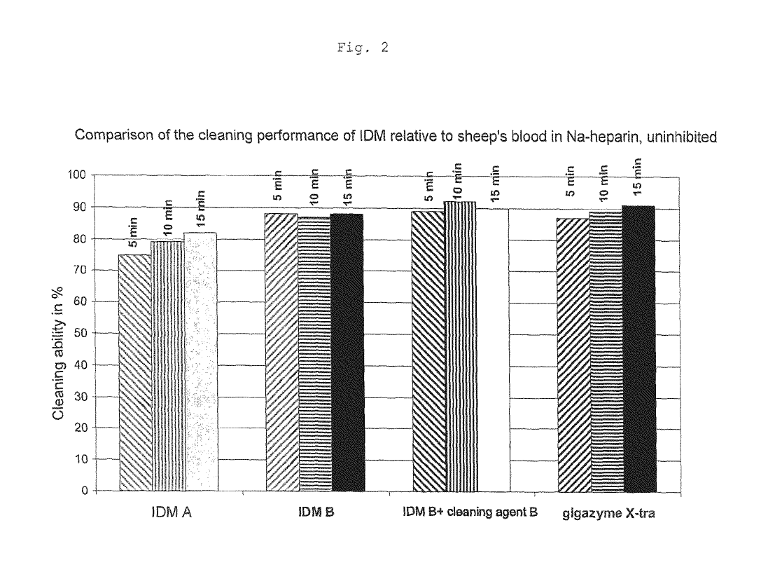

The given concentrations (1% and 1.5%) of the test solutions were produced in mains water and examined after the defined reaction times (5, 10, 15 min). The experiments were performed at room temperature. The following commercially available preparations were used in the investigation (FIG. 2 to FIG. 5):
Cleaning agent A: Bodedex forte (Bode Chemie, Hamburg, Germany)
Cleaning agent B: Mucodont Zymaktiv (Merz Hygiene GmbH, Frankfurt, Germany)
IDM A: Sekusept plus (Ecolab Germany GmbH, Düsseldorf, Germany)
IDM B: Mucocit T (Merz Hygiene GmbH, Frankfurt, Germany)
gigazyme X-tra (Schülke & Mayr GmbH, Hamburg, Germany)

Method

The 100 ml beakers are filled with about 100 ml of the solution to be tested. The slides are placed with the tweezers carefully with the test dirt layer at the top into the solution. At the end of the test period the slides are carefully removed from the solution with the tweezers and rinsed by careful immersion and swirling in VE-water. The slides are then dried upright in air. After about 1 hour the slides are stored for drying overnight in the desiccator using silica gel. Afterwards re-weighing is performed.

Evaluation and Documentation

For the gravimetric evaluation the cleaning performance is calculated in % from the determined weight differences. In addition, there is an optical assessment of the slides and an assessment of the test solution (compared to a freshly prepared one).

Error range

| | Blood | |
|---|---|---|
| Weighing errors | ±0.3 | mg |
| Quantity Ø | 10.0 | mg |
| Error (rel. to quantity Ø) | ±3% | |
| Quantity min. (−20%) | 8.0 | mg |

| | |
|---|---|
| Error (rel. to quantity min) | ±3.8% |

### Results

FIG. 2 shows the comparison of the cleaning performance of different instrument disinfectants according to method II (heparinised sheep's blood). The different formulations were examined according to the recommended application concentrations of 1% (IDM B; gigazyme X-tra) or 1.5% (IDM A) after the indicated reaction times (5, 10, 15 min.). The combination of IDM B+ cleaning agent B was examined at a use concentration of 1% of both components after the indicated reaction times (5, 10, 15 min.). The cleaning performance is indicated in %, where 100% represents the best cleaning result, at which virtually no residual contaminant can be found, which means 100% cleaning of the coagulated blood from the slide. The tests were performed at room temperature as a static test.

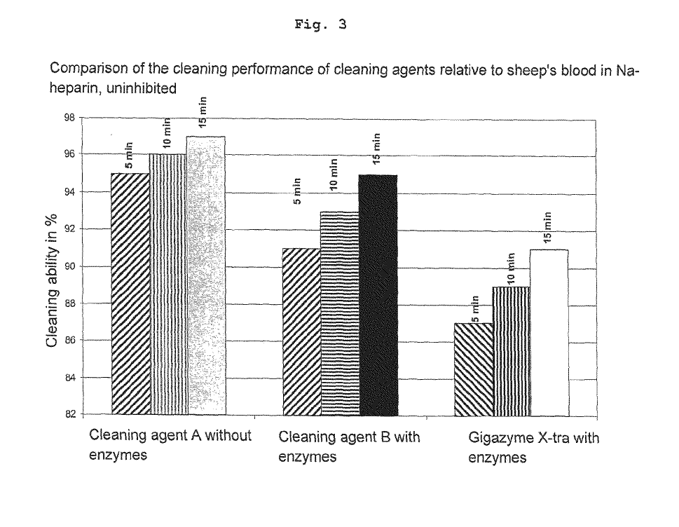

FIG. 3 shows a comparison of the cleaning performance of different cleaning agents for the manual cleaning of medical instruments according to method II (heparinised sheep's blood). The different formulations were according to the recommended application concentrations of 1% (cleaning agent A; cleaning agent B; gigazyme X-tra). The combination of IDM B+ cleaning agent B was examined at a use concentration of 1% respectively after the indicated reaction times (5, 10, 15 min). The cleaning performance is indicated in %, where 100% is the best cleaning result, at which virtually no residual contamination can be found, which means 100% cleaning of the coagulated blood from the slide. The tests were performed at room temperature as a static test.

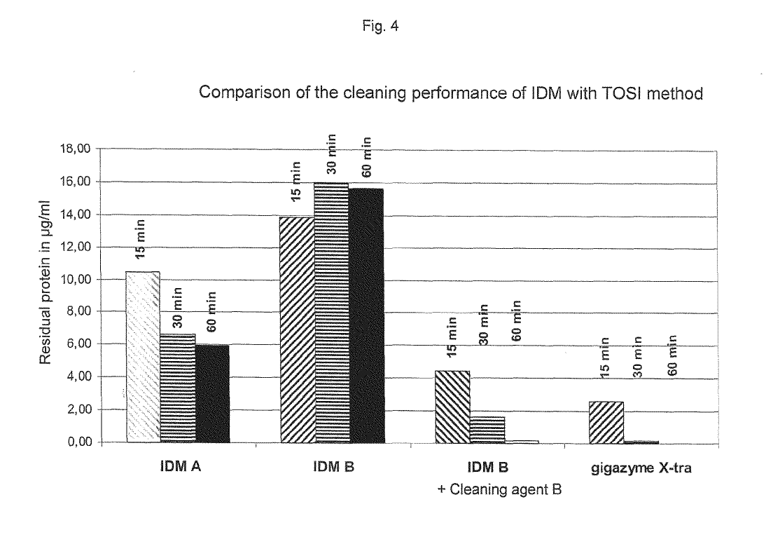

FIG. 4 shows a comparison of the cleaning performance of different instrument disinfectants according to method I (TOSI method). The different formulations were according to the recommended application concentrations of 1% (cleaning agent A; cleaning agent B; gigazyme X-tra) after the indicated reaction times (5, 10, 15 min.). The combination of IDM B+ cleaning agent B was examined at a use concentration of 1% respectively after the indicated reaction times (5, 10, 15 min). The determined residual contamination on the TOSI test bodies after the indicated reaction times is indicated in μg/ml. In this case a high residual contamination means a poor cleaning result and a low value means a low residual contamination. The tests were performed at room temperature as a dynamic test.

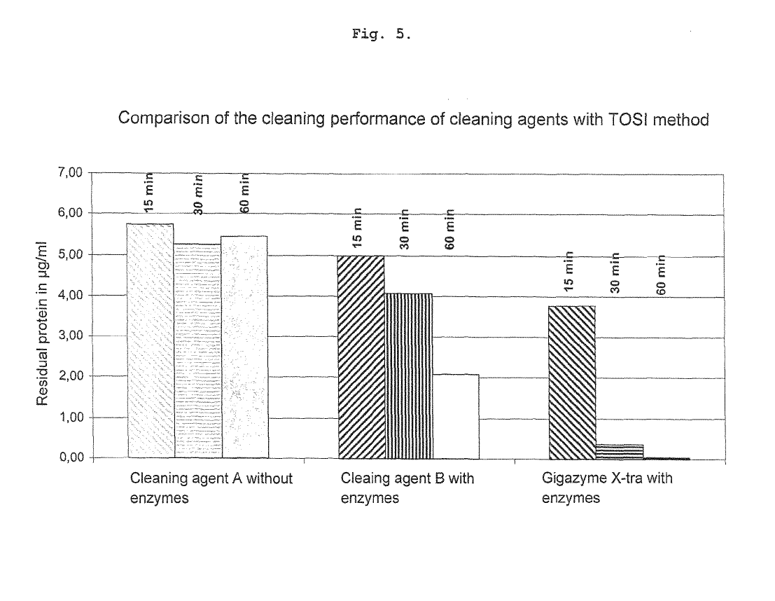

FIG. 5 shows a comparison of the cleaning performance of different cleaning agents for the manual cleaning of medical instruments according to method I (TOSI method). The different formulations were examined according to the recommended application concentrations of 1% (cleaning agent A; cleaning agent B; gigazyme X-tra) after the indicated reaction times (5, 10, 15 min). The combination of IDM B+ cleaning agent B was examined at a concentration of 1% respectively after the indicated reaction times (5, 10, 15 min). The determined residual contamination on the TOSI test bodies after the indicated reaction times is given in μg/ml. In this case a high residual contamination indicates a poor cleaning result and a low value indicates low residual contamination. The investigations were performed at room temperature as a dynamic test.

1. Method for determining the cleaning performance of a formulation, in which
a) the formulation is provided,
b) a test body is brought into contact with the formulation, the test body being contaminated with a protein-containing test contaminant,
c) the contaminated test body is left in contact with the formulation in order to clean the contaminated test body,
d) the cleaned test body is rinsed,
e) if necessary the rinsed test body is dried,
f) if necessary the test contaminant remaining on the test body is evaluated in terms of quality and
g) the test contaminant remaining on the test body is analysed in terms of quantity, the quantitative analysis of the remaining test contamination including the removal of the remaining test contaminant from the test body.

2. Method according to claim 1, wherein the contact c) is a static or a dynamic contact between the formulation and the contaminated test body.

3. Method according to claim 1, wherein the test contaminant of the test body is a synthetic test contaminant, the synthetic test contaminant preferably comprising at least two different protein components, particularly preferably at least three different protein components.

4. Method according to claim 3, wherein the protein component is selected from a group comprising fibrin, a fibrin precursor, haemoglobin and albumin.

5. Method according to claim 1, wherein the rinsing d) includes rinsing with water, wherein the rinsing d) is preferably with water.

6. Method according to claim 1, wherein the drying e) takes place and is performed at a temperature of in the range of 10 to 40° C., preferably in the range of 15 to 30° C., in particular at about 25° C.

7. Method according to claim 1, wherein the qualitative evaluation of the test contaminant remaining on the test body takes place f) and includes an optical evaluation from a scale with at least 10 levels, preferably at least 12 levels, in particular at least 16 levels.

8. Method according to claim 7, wherein the optical evaluation of the test contaminant remaining on the test body is an evaluation in 4 rough levels and the 4 rough levels each comprise four finer levels, and wherein the uncleaned test body with the protein-containing test contaminant represents an additional level and the test body without a protein-containing test contaminant likewise represents an additional level.

9. Method according to claim 1, wherein the qualitative evaluation f) includes photographing the test contaminant remaining on the test body.

10. Method according to claim 1, wherein the qualitative evaluation f) does not include a chemical detection reaction.

11. Method according to claim 1, wherein the test contaminant remaining on the test body is quantified photometrically during the quantitative analysis g) after the removal of the test contaminant, wherein preferably protein contained in the test contaminant remaining on the test body and removed therefrom is quantified photometrically, and wherein in a particularly preferable manner the photometric determination is performed of a protein contained in the test contaminant remaining on the test body and removed therefrom in a buffered solution with a pH of 5 to 9, preferably with a pH of 6 to 8.

12. Method according to claim 1, wherein the removal of the test contaminant remaining on the test body includes rinsing the remaining test contaminant from the test body by means of one or more aqueous solutions.

13. Method according to claim 12, wherein the aqueous solutions used for rinsing consist of an alkaline and an acidic aqueous solution, the remaining test contaminant being rinsed in step g) preferably with an alkaline aqueous solution and then rinsed with an acidic aqueous solution in order to obtain an essentially neutral solution of the test contaminant remaining on the test body.

14. Method according to claim 1, wherein the removal of the test contaminant remaining on the test body includes mechanical cleaning.

15. Method according to claim 11, wherein the indicator for the photometric quantification is a triphenylmethane dye, preferably Coomassie Brilliant Blue G-250.

16. Method according to claim 1, wherein the formulation is an aqueous formulation.

17. Kit for performing the method according to claim 1, which comprises
(i) a set of instructions with a description of the method,
(ii) one or more solutions for removing the remaining contaminant,
(iii) one or more mechanical means for removing the remaining contaminant, the preferred means being glass beads, and
(iv) if necessary a standard series of cleaned test bodies for the qualitative evaluation f), or an image of the standard series.
